(19) United States
(12) Patent Application Publication
(10) Pub. No.: US 2007/0031571 A1
(43) Pub. Date: Feb. 8, 2007

(54) Phytosterol esters

(75) Inventor: Peter A. Blagdon, Erlanger, KY (US)

Correspondence Address: PHILIP L BATEMAN, P O BOX 1105, DECATUR, IL, 62525, US

(73) Assignee: Peter Cremer North America, LP, Cincinnati, OH (US)

(21) Appl. No.: 11/494,667

(22) Filed: Jul. 27, 2006

Related U.S. Application Data
(60) Provisional application No. 60/705,213, filed on Aug. 3, 2005.

(51) Int. Cl. A23D 9/00

(52) U.S. Cl. 426/611

(57) ABSTRACT

A fatty acid phytosterol ester composition wherein at least about 95 percent of the fatty acid chains contain 6, 8, 10, or 12 carbon atoms and wherein at least about 70 percent of the fatty acid chains contain 8 or 10 carbon atoms is water dispersible and especially suited for use in aqueous food compositions.

### CROSS-REFERENCE TO RELATED APPLICATION

This application claims the benefit of U.S. Provisional Application Ser. No. 60/705,213, Aug. 3, 2005.

### FIELD OF THE INVENTION

This invention relates to food compositions. More particularly, this invention relates to water dispersible fatty acid phytosterol ester compositions.

### BACKGROUND OF THE INVENTION

1. Fats

The human diet is predominantly comprised of fats, proteins, and carbohydrates. On a weight basis, fats have nearly twice the energy (typically measured in calories) of proteins and carbohydrates. The term “oil” is sometimes used for fats that are liquid at room temperature. The two terms are used interchangeably herein. Fats are substances of plant or animal origin that consist primarily of triglycerides. A triglyceride is the condensation product of one molecule of glycerol with three molecules of fatty acids to produce one molecule of triglyceride and three molecules of water: 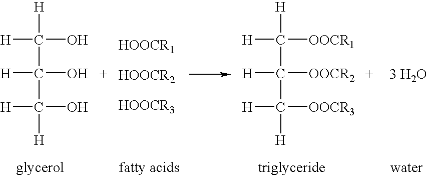
where R₁, R₂, and R₃ represent hydrocarbon groups (also known as chains or radicals). A fatty acid is an aliphatic compound containing 4 to 24 carbon atoms and having a terminal carboxyl group (—COOH). A triglyceride is a member of the class of organic compounds known as esters in that the hydrogens of the terminal carboxyl groups of the fatty acids are exchanged for an organic radical. Naturally occurring fats contain small quantities of a variety of other compounds, including free fatty acids, glycerol, and sterols. Fats are frequently refined to remove these compounds.

Naturally occurring fatty acids, with only minor exceptions, have an even number of carbon atoms and, if any unsaturation is present, the first double bond is generally located between the ninth and tenth carbon atoms. Fatty acids with a single double bond are sometimes called monounsaturated. Fatty acids with two or more double bonds are sometimes called polyunsaturated. Fatty acids are sometimes abbreviated by listing their number of carbon atoms and their number of double bonds. Some of the more common fatty acids in naturally occurring fats are listed in Tables 1 and 2.

TABLE 1
SATURATED FATTY ACIDS

| Common Name | Abbreviation | Formula |
|---|---|---|
| Caproic | 6:0 | C₆OOH |
| Caprylic | 8:0 | C₈OOH |
| Capric | 10:0 | C₁₀OOH |
| Lauric | 12:0 | C₁₂OOH |
| Myristic | 14:0 | C₁₄OOH |
| Palmitic | 16:0 | C₁₆OOH |
| Stearic | 18:0 | C₁₈OOH |

TABLE 2
UNSATURATED FATTY ACIDS

| Common Name | Abbreviation | Formula |
|---|---|---|
| Caproleic | 10:1 | C═C₉OOH |
| Lauroleic | 12:1 | C₃═C₉OOH |
| Oleic | 18:1 | C₉═C₉OOH |
| Linoleic | 18:2 | C₆═C₃═C₉OOH |
| Linolenic | 18:3 | C₃═C₃═C₃═C₉OOH |
| Eicosenoic | 20:1 | C₁₁═C₉OOH |
| Erucic | 22:1 | C₁₃═C₉OOH |

The distribution of the different fatty acid radicals varies among naturally occurring fats. The distribution of free fatty acids is the same as the distribution of fatty acid radicals in the triglycerides. For example, the distribution of fatty acids in soybean oil triglycerides is typically about 55% linoleic, 22% oleic, 11% palmitic, 8% linolenic, 3% stearic, and 1% other. Thus, soybean oil has an unsaturation level of about 85% (i.e., it has about 85% unsaturated fatty acids and about 15% saturated fatty acids). In contrast, the distribution of fatty acids in rapeseed oil is typically about 50% erucic, 15% oleic, 15% linoleic, 8% linolenic, 5% eicosenoic, 2% stearic, 2% palmitic, and 3% other. Thus, rapeseed oil has an unsaturation level of about 93%. The rapeseed plant has been bred to change the distribution of the fatty acids in its oil. The oil from the modified breed of plant is known as canola oil. Canola oil contains less erucic acid and more oleic, linoleic, and linolenic acids.

The ratio of unsaturated fatty acids to saturated fatty acids affects many properties of the fat. Unsaturated fatty acids are known to be more digestible and are more beneficial after digestion to humans and animals than saturated fatty acids. An unsaturated fatty acid also has a lower melting point than a saturated fatty acid of the same number of carbon atoms. For example, animal fats have a relatively high percentage of saturated fatty acids and are typically solids at room temperature. In contrast, vegetable oils have a relatively high percentage of unsaturated fatty acids and are typically liquids at room temperature.

Although unsaturated fatty acids are believed to have many health advantages over saturated fatty acids, they also have some physical properties that cause problems in food use. As mentioned above, the unsaturated fatty acids have lower melting points and certain applications require a fat with a higher melting point. Another undesirable property of unsaturated fatty acids is their reactivity with atmospheric oxygen. This oxidation produces a variety of decomposition products, some of which have unpleasant odors. A fat that has oxidized and developed an unpleasant odor is said to be “rancid.”

Unsaturated fatty acids can be converted to saturated fatty acids by a process known as hydrogenation. Hydrogenation raises the melting point and improves the shelf life of the fat. However, hydrogenation is believed to have an adverse dietary effect. Not only are saturated fatty acids less healthy than unsaturated fatty acids, hydrogenation also rearranges some of the double bonds. Double bonds are described as cis- or trans- depending on the spatial arrangement of the molecule on either side of the bond. Naturally occurring fatty acids are generally in the cis- form. Hydrogenation converts some cis- form molecules into the trans- form. A trans- linkage produces less irregularity in the straight-chain structure and results in a slightly higher melting point and less reactivity. It is believed that fats containing trans- form fatty acids are especially deleterious to the health.

2. Phytosterols

Steroids are polycyclic compounds having a nucleus of four cyclic rings comprising seventeen carbon atoms fused together. The ring structure typically contains one carbon-carbon double bond. A variety of hormones, bile acids, and vitamin precursors are steroids. Most steroids have two methyl groups (—CH₃) attached to the nucleus. Most steroids also have an aliphatic side group attached to the nucleus.

Sterols are steroids that contain a hydroxyl group (—OH). The aliphatic side chain of sterols typically contains eight to ten carbon atoms. Phytosterols are sterols derived from plant sources. The chemical structure of a phytosterol is shown in FIG. 1 where R₂ is the aliphatic side chain. As mentioned above, phytosterols are removed from the triglycerides during the refining of vegetable oils. Phytosterols can be hydrogenated to remove the carbon-carbon double bond. Such hydrogenated compounds are sometimes referred to as stanols, but the term “phytosterol” is used herein to include such hydrogenated compounds

Phytosterols in the diet are believed to reduce benign prostatic hyperplasia (the enlargement of the prostate gland). Phytosterols are also believed to reduce hypercholesteremia (high cholesterol in the bloodstream which, in turn, is believed to cause various problems in the circulatory system). The U.S. Food and Drug Administration has authorized food manufacturers to designate products as being healthy for the heart if they contain a certain level of sterols. Accordingly, there is now an incentive to add phytosterols to foods. Phytosterols have been added to a variety of foods, including margarine and yogurt.

Unfortunately, phytosterols have extremely low solubilities in water and very low dispersibilities in water. This greatly limits the types of foods to which phytosterols can be added. Several different approaches have been employed to add phytosterols to aqueous (water containing) food compositions.

One approach is to first form fatty acid sterol esters. The fatty acids used to produce the esters are generally derived from vegetable oils such as soybean, canola, rapeseed, sunflower seed, palm, cottonseed, and the like. These esters are then blended with suitable emulsifiers and mixed with the water composition to form an oil-in-water emulsion. Unfortunately, a different emulsion system is required for each composition. Creating a stable emulsion also requires the use of highly sophisticated mixing systems and/or large amounts of emulsifiers.

A second approach is to spray dry the fatty acid sterol esters into a readily soluble/dispersible matrix such as corn syrup solids with an emulsifier. Relatively small quantities of this material can be dispersed in water. Spray drying into a matrix is relatively expensive and the spray dried esters have a high percentage of non-sterol ingredients.

A third approach is to add finely ground free sterols to the aqueous composition. Unless the sterols are ground to an extremely small particle size, they give the food a “chalky” mouthfeel. Grinding sterols to such small particle sizes is an expensive and difficult process.

These three approaches are relatively expensive and use phytosterol compositions that have limited water dispersibilities and that are not suitable for all food compositions. Accordingly, there is a demand for an improved water dispersible phytosterol composition that is especially suited for aqueous food compositions.

### SUMMARY OF THE INVENTION

One general object of this invention is to provide an improved water dispersible phytosterol composition. A more particular object is to provide a phytosterol composition with increased water dispersibility and that is especially suited for aqueous food compositions. Other objects are to provide a food composition containing a phytosterol and to provide an improved method of making phytosterols.

I have invented a water dispersible phytosterol composition. The composition comprises fatty acid phytosterol esters wherein at least about 95 percent of the fatty acid chains contain 6, 8, 10, or 12 carbon atoms and wherein at least about 70 percent of the fatty acid chains contain 8 or 10 carbon atoms.

I have also invented a food composition. The composition comprises water and a water dispersible fatty acid phytosterol ester composition wherein at least about 95 percent of the fatty acid chains contain 6, 8, 10, or 12 carbon atoms and wherein at least about 70 percent of the fatty acid chains contain 8 or 10 carbon atoms.

I have further invented a method of making a water dispersible fatty acid phytosterol ester composition. The method comprises reacting fatty acids with a phytosterol wherein at least about 95 percent of the fatty acids contain 6, 8, 10, or 12 carbon atoms and wherein at least about 70 percent of the fatty acids contain 8 or 10 carbon atoms.

The phytosterol composition of this invention is water dispersible and is especially suited for use in a wide variety of aqueous food compositions.

### BRIEF DESCRIPTION OF THE DRAWINGS

FIG. 1 is a diagram showing a single-step process for making the fatty acid phytosterol esters of this invention.

FIG. 2 is a diagram showing a two-step process for making the fatty acid phytosterol esters of this invention.

### DETAILED DESCRIPTION OF THE INVENTION

1. The Invention In General

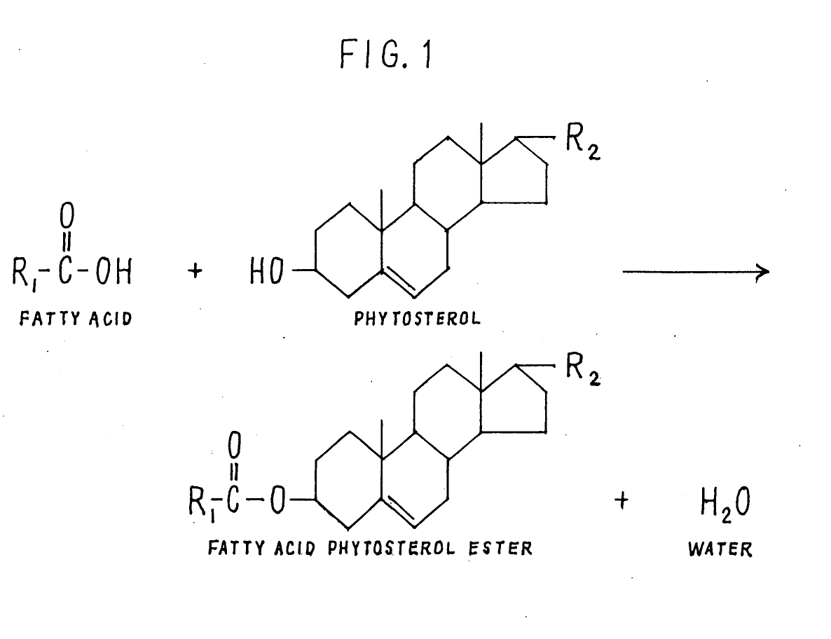

The fatty acid phystosterol esters of this invention can be produced by reacting a fatty acid with a phytosterol as shown in FIG. 1 where R₁ and R₂ represent aliphatic hydrocarbon chains. As explained in detail below, R₁ preferably contains seven or nine carbon atoms (giving the fatty acid a total of eight or ten carbon atoms when the carbon atom of the —COOH carboxyl group is added) and R₂ represents an aliphatic hydrocarbon chain that generally contains eight to ten carbon atoms. R₁ most preferably contains either seven or nine carbon atoms, but not both.

2. The Fatty Acid Component

The fatty acid component of the ester is typically derived from naturally occurring fats. For use in this invention, at least about 95 percent of the fatty acids contain 6, 8, 10, or 12 carbon atoms and at least about 70 percent of the fatty acids contain 8 or 10 carbon atoms. The fatty acid component may be mixtures of fatty acids or may be a pure fatty acid. Preferably, at least about 80 percent of the fatty acids contain 8 or 10 carbon atoms and, more preferably, at least about 90 percent of the fatty acids contain 8 or 10 carbon atoms. Preferably, at least about 90 percent of the fatty acids are saturated. Thus, the preferred fatty acid component contains at least about 90 percent of either caprylic acid (C₈OOH), capric acid (C₁₀OOH), or a mixture of the two.

Preferred fatty acid sources are coconut or palm kernel oil fatty acids from which the longer chains have been removed. These sources contain about 90 to 97 percent caprylic and capric fatty acids. The balance of the fatty acids are substantially caproic (C₆OOH) and lauric (C₁₂OOH) fatty acids.

When mixtures of fatty acids are used, the resulting phytosterol esters have melting points that are below that of esters formed from a pure fatty acid such as caprylic acid or capric acid. In some food compositions, a higher melting point is desirable and/or essential. Solutions of two compounds have a lower melting point than either compound by itself. Accordingly, relatively pure caprylic acid esters or relatively pure capric acid esters are advantageously used in such food compositions.

Relatively pure caprylic acid esters or relatively pure capric acid esters can be obtained in two basic ways. First, they can be obtained by starting with a relatively pure source of caprylic acid or capric acid (typically greater than about 90, preferably greater than about 95 percent, and most preferably greater than about 99 percent). Relatively pure caprylic acid and relatively capric acid are typically isolated by vacuum distillation of fatty acids. Alternatively, relatively pure caprylic acid esters or relatively pure capric acid esters can be obtained by isolating them from a mixture of esters. This isolation is described in more detail below.

3. The Phytosterol Component

The phytosterol component of the ester is an article of commerce. As previously mentioned, the term “phytosterol” includes stanols. Phytosterols are commercially available from a large number of oilseed refiners and other suppliers. Phytosterols are typically recovered from the deodorizer (a distillation colunm operated at a vacuum) distillate by oilseed refiners. Phytosterols are also commercially available from paper manufacturers. These phytosterols are separated from the tall oil, which is a mixture of compounds obtained from the treatment of liquids from the digesting of pine wood.

4. Reaction

As previously mentioned, the fatty acids and phytosterol can be reacted directly to produce fatty acid phytosterol esters as shown in FIG. 1. However, the direct reaction is generally not performed for two reasons. First, the reaction is usually driven by an excess of the fatty acid component that must be removed by distillation after the reaction is completed. The fatty acid ester has a boiling point substantially less than the free fatty acid and is easier to remove after the reaction has finished. Second, the reaction is catalyzed by an alkaline catalyst. If the direct esterification with a fatty acid is attempted the alkaline catalyst reacts with the free fatty acids to form soaps and the reaction slows or stops. In addition this reaction generates water that forms a gummy residue with the soaps.

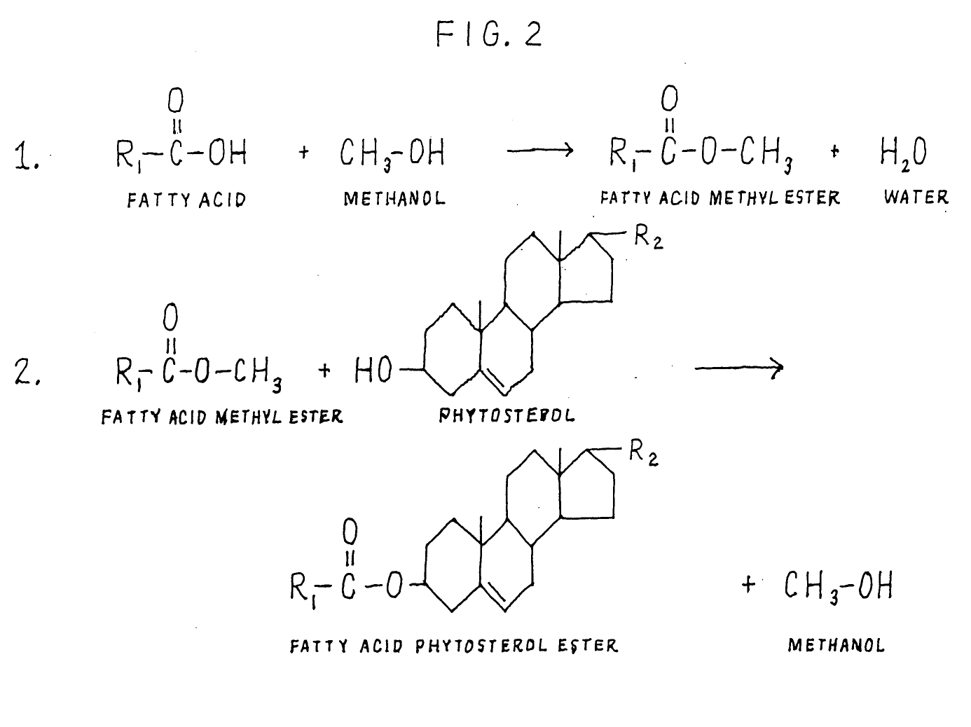

A variety of other pathways are available for producing the fatty acid phytosterol esters. A preferred pathway is the two-step sequence illustrated in FIG. 2. The first step in preparing a mixed fatty acid phytosterol ester is to conduct an esterification reaction as follows. A mixture of free fatty acids and glycerol derived from the refining of a liquid triglyceride (such as coconut oil or palm kernel oil) are mixed with a 5 molar excess of methanol (or other aliphatic alcohol) and an alkaline catalyst such as alcoholic potassium hydroxide or a solution of sodium methoxide in methanol. The mixture is agitated and heated under reflux for several hours. The free fatty acids react with the methanol to produce fatty acid methyl esters and water. The reaction mixture is allowed to separate into two layers. The upper layer contains the fatty acid methyl esters and most of the excess methanol. The lower level contains primarily glycerol. The glycerol portion is decanted and the upper level is washed with acidulated water to remove excess catalyst, excess methanol, and residual glycerol. The resulting fatty acid methyl ester is dried by heating under a mild vacuum.

A fractional distillation under vacuum is then conducted to concentrate the caprylic and capric methyl esters. The heavier fractions (enriched in lauric and myristic fatty acid methyl esters) are utilized for other purposes.

The second step of the preferred pathway is to conduct a transesterification reaction as follows. A reaction mixture is prepared using about a 3 molar excess of the caprylic and capric enriched fatty acid methyl esters with crystalline sterols and an alkaline catalyst (typically sodium methoxide). The reaction mixture is heated and stirred at a temperature such that the sterols dissolve in the fatty acid methyl ester. As the reaction proceeds, the methanol evolved by the reaction is stripped off to drive the reaction. Finally the reaction is stopped by the addition of a mild acid and then the remaining unreacted fatty acid methyl ester is removed by high vacuum (10 millibars or less) thin layer distillation leaving the fatty acid phytosterol esters.

As mentioned above, it is often desirable to isolate relatively pure caprylic acid phytosterol esters or relatively pure capric acid phytosterol esters because the pure compounds have a higher melting point. A preferred method of isolating these phytosterol esters is to form the methyl esters from a mixture of fatty acids. The caprylic acid methyl esters and the capric acid methyl esters are then separately isolated by fractional distillation. The pure methyl esters are then converted to the pure phytosterol esters by the transesterification reaction.

5. Further Processing

The mixed phytosterol esters are waxy solids having a melting point slightly above room temperature. For example a 50-50% mixture of caprylic and capric acid phytosterol esters has a melting point of about 54° C. When cooled, they congeal relatively slowly and remain tacky for several hours at room temperature. After congealing, the mass can be broken into small pieces and dispersed or suspended in the aqueous liquid without the need for emulsifiers or sophisticated mixing equipment.

The phytosterol esters can also be converted into a fine powder before use. The first step of this conversion is to break the congealed mass into smaller pieces. The small pieces are then cooled to a temperature well below the freezing point of water, preferably to a temperature below about −50° C. One way of achieving this cooling is by mixing the particles with dry ice. Dry ice is solid carbon dioxide which sublimates (changes directly from the solid phase to the gas phase) at atmospheric pressure at a temperature of −79° C. This cooling is also achievable in many other ways known to the art, including immersion in liquid nitrogen which has a normal boiling point of −195° C.

The cooled particles are then placed into a blender with chunks of dry ice or other cryogenic medium to maintain the low temperature. After grinding, the resulting powder is tempered by warming to a temperature above the dew point of the ambient air while the gas from the evaporating cryogenic medium blankets the powder to prevent condensation of moisture from the ambient air. The powder is then mixed with a small amount of a free flow agent such as CAB-O-SIL silica to form a free flowing powder. The powder is then screened to the desired size. The powder generally has a particle size of less than about 250 microns and preferably has a particle size less than about 100 microns.

Other processes for making oleaginous solids into particles, such as flaking, pastillating, prilling, and spray chilling, are not effective with the mixed phytosterol esters.

Pure caprylic acid phytosterol esters have a melting point of about 73° C. and pure capric acid phytosterol esters have a melting point of about 78° C. These melting points are high enough that the pure esters can be flaked without cryogenic cooling. If desired, the flakes can then be cryoground or otherwise treated.

6. Use of the Composition

The fatty acid phytosterol esters of this invention can be dispersed or suspended in aqueous liquids without the need for emulsifiers or sophisticated mixing equipment. The resulting suspensions are often clear or translucent. The suspensions have a relatively bland taste with a creamy mouthfeel. Accordingly, the esters are suitable for addition to a wide variety of aqueous food compositions, including cold beverages (such as orange juice), hot beverages (such as hot chocolate and soups), cooked sauces (such as gravies), desserts (such as chocolate and puddings), fat based spreads (such as margarine and peanut butter), uncooked foods, fruit fillings and related products, and dairy products such as milk beverages, ice cream, ice milk, sherbets, yogurts, and smoothies. As previously mentioned, the addition of the fatty acid phytosterols is believed to reduce benign prostatic hyperplasia (the enlargement of the prostate gland) and to reduce hypercholesteremia (high cholesterol in the bloodstream which, in turn, is believed to cause various problems in the circulatory system).

The mixed fatty acid phytosterol esters of this invention contain about 70 percent sterol on a weight basis. A pure caprylic acid phytosterol ester contains about 74 percent sterol on a weight basis. In contrast, a phytosterol ester made from soybean or canola fatty acids would contain about 60 percent sterol. Accordingly, the phytosterol esters of this invention provide a greater amount of sterol per unit of weight.

7. Examples

The following examples are illustrative only.

### EXAMPLE 1

This example illustrates the preparation of a water dispersible mixed phytosterol composition. A phytosterol ester is prepared using the two-step sequence described above. The congealed mass is broken into small particles. The phytosterol esters contain 72 weight percent sterol and are more than 90 percent a mixture of caprylic acid esters and capric acid esters.

### EXAMPLE 2

The mixed phytosterol esters of Example 1 are converted into powder form using dry ice, a blender, and a screen as described above. The powder has a particle size of less than 250 microns.

### EXAMPLE 3

This example illustrates the use of the mixed phystosterol composition of Example 1 in a hot chocolate composition.

Four trials were conducted using NESTLE hot chocolate mix in individual 15 gram packets. Each trial was conducted with 18 ounces of hot water at approximately 180° F. and three packets of the hot chocolate mix. In each trial, the blender was preheated by filling half full with hot water, covering the container, running the blender for 30 seconds, and then pouring out the hot water. Preheating helps to prevent the phytosterols from smearing on the inside of the container during mixing. The control trial contained no sterol. The three experimental trials each contained 400 mg of sterol per 6 ounce serving of hot chocolate.

The first trial was conducted as a control by placing the hot water and the hot chocolate mix in a covered WARING blender. The blender was run at high speed for 30 seconds. The blender was then turned off and the mixture was tasted by a panel. The mixture was evaluated as a pleasant hot chocolate beverage.

The second trial was the same as the first except 4.08 grams of a spray dried sterol ester were also added. The mixture was evaluated as a pleasant hot chocolate beverage.

The third trial was the same as the first except 1.20 grams of free sterols ground to pass a United States Screen #140 screen (105 microns) were also added. The mixture was evaluated as having a slightly gritty or chalky mouthfeel.

The fourth trial was the same as the first except 1.74 grams of the phytosterol esters of Example 1 were also added. The mixture was evaluated as a pleasant hot chocolate beverage.

### EXAMPLE 4

This example illustrates the use of the mixed phytosterol composition of Example 1 in milk.

Two hundred fifty milliliters (250 ml) of two percent (2%) fat milk and 1.5 grams of the phytosterol esters of Example 1 were placed into a covered WARING blender. The blender was run at medium speed for about 30 seconds. The blender was stopped and the mixture was allowed to hydrate for about five minutes. The blender was then run at medium speed for another 30 seconds.

The fortified milk had a creamy, pleasant texture and the phytosterol esters remained stable in the milk. The fortified milk is suitable for further processing into dairy products such as ice cream, yogurt, and the like.

### EXAMPLE 5

This example illustrates the use of the mixed phytosterol composition of Example 2 in a chocolate pudding.

3.4 ounce package of JELLO chocolate pudding was blended with one cup of whole milk in a saucepan and then heated to boiling. As soon as the product thickened, it was removed from heat and 1.3 grams of the phytosterol powder were stirred into the mix. The mix was placed in individual covered bowls and refrigerated for 24 hours. The phytosterols were uniformly suspended in the pudding.

### EXAMPLE 6

This example illustrates the use of the mixed phytosterol powder of Example 2 in orange juice.

Twenty-four ounces (24 oz.) of refrigerated orange juice were poured into a blender. As the blender was operated at low speed, 1.95 grams of the phytosterol powder were added. The blender was run for an additional 30 to 40 seconds and then stopped. The resulting orange juice blend had a creamy appearance due to the entrapped air. As the air was released, the orange juice acquired a normal appearance. Some particles of hydrated phytosterols floated at the top of the liquid. After about 15 minutes, the orange juice blend was reblended. The hydrated particles were reduced in size and were less apparent in the finished orange juice.

### EXAMPLE 7

This example compares the physical properties of mixed phytosterol esters with pure caprylic phytosterol esters and pure capric phytosterol esters.

A mixture of 60 percent caprylic and 40 percent capric phytosterol esters was melted by heating it to 90° C. The melted mixture was then poured onto a metal sheet at a temperature of 20° C. After two hours, the mixture of esters was still sticky, was clear, and could not be scraped from the sheet.

A mixture of 10 percent caprylic and 90 percent capric phytosterol esters was melted by heating it to 90° C. The melted mixture was then poured onto a metal sheet at a temperature of 20° C. The mixture of esters congealed in about 25 seconds, exhibited opaque solids formation, and could be readily scraped from the sheet.

A pure caprylic acid phytosterol ester was melted by heating it to 90° C. The liquid was then poured onto a metal sheet at a temperature of 20° C. The pure esters congealed in about 20 seconds, exhibited opaque solids formation, and could be readily scraped from the sheet.

A pure capric acid phytosterol ester was melted by heating it to 90° C. The liquid was then poured onto a metal sheet at a temperature of 20° C. The pure esters congealed in about 20 seconds, exhibited opaque solids formation, and could be readily scraped from the sheet.

### EXAMPLE 8

This example compares the use of mixed phytosterol esters with pure caprylic phytosterol esters in a liquid hot chocolate composition.

A first hot chocolate composition was prepared as follows. Six hundred milligrams (600 mg) of a mixture of 60 percent caprylic and 40 percent capric phytosterol esters was dry blended with one 15 gram packet of NESTLE hot chocolate mix. One hundred eighty grams (180 g) of water at 65° C. was added and mixed. The resulting hot chocolate was slightly gummy.

A second hot chocolate composition was prepared as above except 600 hundred milligrams of pure caprylic phytosterol esters were used in place of the mixture. The resulting hot chocolate was not gummy, was very palatable, and did not chum out.

### EXAMPLE 9

This example compares the use of mixed phytosterol esters with pure caprylic phytosterol esters in a solid milk chocolate composition.

A first solid milk chocolate composition was prepared as follows. One hundred twenty grams (120 g) of NESTLE solid milk chocolate was melted in a double boiler. Six grams (6 g) of a mixture of 60 percent caprylic and 40 percent capric phytosterol esters were blended into the melt. The melted material was poured into an aluminum pan and cooled for three hours at room temperature. The resulting chocolate had solidified but was still soft. The chocolate had a slightly greasy mouth feel.

A second solid milk chocolate composition was prepared as above except six grams of pure caprylic phytosterol esters were used in place of the mixture. The resulting solid chocolate was completely hardened, had a good snap when broken, and had a pleasant mouth feel.

### EXAMPLE 10

This example compares the use of two mixed phytosterol esters with pure caprylic phytosterol esters in a solid dark chocolate composition.

A first solid dark chocolate composition was prepared as follows. Thirty seven and one-half grams (37.5 g) of DOVE dark chocolate was melted by placing it in an oven at 85° C. for 30 minutes. Four and seven-tenths grams (4.7 g) of a commercial phytosterol ester composition containing 60 percent sterol were blended into the melted chocolate. The resulting mixture contained 7.5 grams sterol per 100 grams chocolate.

The mixture was then placed in a room at a constant temperature of 100° F. for 19 hours. The mixture was then moved to a room at ambient conditions for two days for cooling. The mixture had solidified, but had a very soft texture. It exhibited no bloom.

A second solid dark chocolate composition was prepared as above except 3.9 grams of a mixture of 60 percent caprylic and 40 percent capric phytosterol esters were blended into the melt. The phytosterol composition contained 72 percent sterol and the resulting mixture contained 7.5 grams sterol per 100 grams chocolate. After cooling, the mixture had good snap and firmness. It exhibited considerable bloom.

A third solid dark chocolate composition was prepared as above except 3.6 grams of pure caprylic phytosterol esters were blended into the melt. The phytosterol composition contained 76 percent sterol and the resulting mixture contained 7.5 grams sterol per 100 grams chocolate. After cooling, the mixture had good snap and firmness. It exhibited a very small level of bloom.

I claim:

1. A water dispersible fatty acid phytosterol ester composition wherein at least about 95 percent of the fatty acid chains contain 6, 8, 10, or 12 carbon atoms and wherein at least about 70 percent of the fatty acid chains contain 8 or 10 carbon atoms.

2. The composition of claim 1 wherein at least about 90 percent of the fatty acid chains are saturated.

3. The composition of claim 2 wherein at least about 90 percent of the fatty acid chains contain 8 or 10 carbon atoms.

4. The composition of claim 3 wherein at least about 90 percent of the fatty acid chains contain 8 carbon atoms.

5. The composition of claim 3 wherein at least about 90 percent of the fatty acid chains contain 10 carbon atoms.

6. A food composition comprising water and a water dispersible fatty acid phytosterol ester composition wherein at least about 95 percent of the fatty acid chains contain 6, 8, 10, or 12 carbon atoms and wherein at least about 70 percent of the fatty acid chains contain 8 or 10 carbon atoms.

7. The composition of claim 6 wherein at least about 90 percent of the fatty acid chains are saturated.

8. The composition of claim 7 wherein at least about 90 percent of the fatty acid chains contain 8 or 10 carbon atoms.

9. The composition of claim 8 wherein at least about 90 percent of the fatty acid chains contain 8 carbon atoms.

10. The composition of claim 8 wherein at least about 90 percent of the fatty acid chains contain 10 carbon atoms.

11. The composition of claim 6 wherein the food comprises cold beverages, hot beverages, cooked sauces, desserts, fat based spreads, uncooked foods, fruit fillings, milk beverages, ice cream, ice milk, sherbets, yogurts, or smoothies.

12. A method of making a water dispersible fatty acid phytosterol ester composition comprising reacting fatty acids or aliphatic alcohol esters of fatty acids with a phytosterol wherein at least about 95 percent of the fatty acids contain 6, 8, 10, or 12 carbon atoms and wherein at least about 70 percent of the fatty acids contain 8 or 10 carbon atoms.

13. The method of claim 12 wherein at least about 90 percent of the fatty acids are saturated.

14. The method of claim 13 wherein at least about 90 percent of the fatty acid chains contain 8 or 10 carbon atoms.

15. The method of claim 14 wherein in the phytosterol esters are cooled to a temperature below about −50° C. and then ground in contact with a cryogenic medium to a particle size of less than about 250 microns.

16. The method of claim 15 wherein the ground phytosterol esters are tempered under a blanket of inert gas to exclude moisture from the ambient air until they reach a temperature above the dew point of the ambient air to prevent condensation.

17. The method of claim 16 wherein the tempered phytosterol esters are mixed with a free flow agent and are then screened.

18. The method of claim 14 wherein at least about 90 percent of the fatty acid chains contain 8 carbon atoms.

19. The method of claim 14 wherein at least about 90 percent of the fatty acid chains contain 10 carbon atoms.

20. The method of claim 12 additionally comprising adding the phytosterol esters to a food composition comprising cold beverages, hot beverages, cooked sauces, desserts, fat based spreads, uncooked foods, fruit fillings, milk beverages, ice cream, ice milk, sherbets, yogurts, or smoothies.
